(19) United States
(12) Patent Application Publication
(10) Pub. No.: US 2014/0206941 A1
(43) Pub. Date: Jul. 24, 2014

(54) Systems and Methods for Surgical Access to Delicate Tissues

(71) Applicant: Osteomed LLC, Addison, TX (US)

(72) Inventors: Eric Stiner, North Tustin, CA (US); Thomas Purcell, Henderson, NV (US); Bojan Gospavic, Plano, TX (US); Alan Adam Klompus, Carrollton, TX (US); Thomas Andrew Schmitt, Addison, TX (US)

(73) Assignee: Osteomed LLC, Addison, TX (US)

(21) Appl. No.: 14/142,554

(22) Filed: Dec. 27, 2013

Related U.S. Application Data
(60) Provisional application No. 61/747,089, filed on Dec. 28, 2012.

(51) Int. Cl. A61M 39/02; A61B 17/02

(52) U.S. Cl. CPC A61M 39/02; A61B 17/0218
USPC 600/205; 29/428

(57) ABSTRACT

Surgical instruments providing access to delicate tissue, such as brain tissue, through a transcutaneous channel. A surgical access assembly has a pin component removably fastened to a cannula component such that rotational forces are translated between the pin component and the cannula component. The pin component is adapted to receive and secure a guide-pin component from a navigation system to assist in the proper initial placement of the surgical access assembly. The cannula component has features on the outer surface configured to facilitate insertion and retention of the surgical access assembly in the target delicate tissue. When desired, pin component can be removed from cannula component to reveal an inner cavity configured to provide access to the target delicate tissue and a working channel for the particular procedure to be performed.

### CROSS-REFERENCE TO RELATED APPLICATIONS

The present application claims priority on U.S. Provisional Application No. 61/747,089, filed Dec. 28, 2012 and entitled “SYSTEMS AND METHODS FOR SURGICAL ACCESS TO DELICATE TISSUES,” the disclosure of which is incorporated herein by reference in its entirety.

### TECHNICAL FIELD

Embodiments of the present invention relate generally to apparatus and techniques to surgery of delicate tissues, and more particularly to apparatus and techniques that provide surgical access to delicate tissues, including trajectory guidance for insertion of medical devices into such tissue.

### BACKGROUND

Delicate tissues, such as brain tissue, are soft and delicate. In particular, the brain tissue is a gel-like substance that can be easily damaged. As such, surgical procedures performed on such delicate tissues demand special considerations. In particular, obtaining surgical access to the brain typically requires creating an opening in the skull, and inserting one or more instruments, commonly known as retractors, to pull back the brain tissue and provide access to certain locations within the brain.

Traditional surgical brain retractors are thin, firm or malleable bands of steel or other metal alloys, with abrupt or well-defined edges and have limited surface areas. These traditional retractors can be introduced into the tissue of the brain or along brain surfaces, and then pulled with force to either separate or elevate the brain tissue during surgery. This method allows the target area to be illuminated and visualized in order to perform the surgical procedure. Given the nature of delicate tissue, a complication known as “retraction injury” can occur, sometimes resulting in compromised brain function. Further, the brain tissues can be torn by the relatively sharp edges of these retractors, and/or the retracted brain can lose blood supply when the local pressure beneath the retractor is greater than venous pressure.

The combination of factors including the softness of the brain tissue, and the effects of sharp, blunt edges and limited surface area of traditional metal band retractor also results in limited visualization of the surgical target area. For instance, the brain tends to extend beyond or “droop” around the edges of the retractor, limiting the area necessary for lighting and reducing overall visibility.

While the retractor described in US Publication Application No. 2010/0010315 addresses the effects of sharp, blunt edges of traditional metal band retractors, it contains small and complicated mechanisms that are difficult to assemble and use during surgical procedures. Further, it does not allow for secured coupling with a navigation system to facilitate a precise initial placement of the retractor. Inaccurate placement can lead to extended procedural time and potential damage to the surrounding structures.

### BRIEF SUMMARY

Embodiments of the present invention provide a device and method for the retraction of tissues to provide a working channel for performing surgery. The surgical access assembly of the present invention is particularly applicable to perform surgery on delicate tissues, such as brain and breast tissues, although it may be used in any medical context. In one embodiment, the surgical access assembly of the present invention can be used for the insertion of surgical instruments. Embodiments of the surgical access assembly of the present invention enable surgery to be performed in a minimally invasive manner, making possible a shorter recovery period for the patient. Certain embodiments of the surgical instrument assembly also allow for integration with navigation computer guidance systems to improve placement of surgical instruments and reduce risk of damages to the delicate tissue.

In one embodiment, a surgical access assembly may have a pin component removably fastened to a cannula component such that rotational forces are translated between the pin component and the cannula component. The pin component is adapted to receive and secure a guide-pin component from a navigation system to assist in the proper initial placement of the surgical access assembly. The cannula component may have features on the outer surface configured to facilitate insertion and retention of the surgical access assembly in the target delicate tissue. When desired, pin component can be removed from cannula component to reveal an inner cavity configured to provide access to the target delicate tissue and a working channel for the particular procedure to be performed.

The foregoing has outlined rather broadly the features and technical advantages of the present invention in order that the detailed description that follows may be better understood. Additional features and advantages will be described hereinafter which form the subject of the claims. It should be appreciated by those skilled in the art that the conception and specific embodiment disclosed may be readily utilized as a basis for modifying or designing other structures for carrying out the same purposes of the present application. It should also be realized by those skilled in the art that such equivalent constructions do not depart from the spirit and scope of the application as set forth in the appended claims. The novel features which are believed to be characteristic of embodiments described herein, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present embodiments.

### BRIEF DESCRIPTION OF THE DRAWINGS

For a more complete understanding, reference is now made to the following descriptions taken in conjunction with the accompanying drawings, in which:

FIG. 1 is a perspective side view of exemplary embodiments of the surgical access assembly according to aspects of the present invention;

FIG. 2A is a cross section of the pin component connected to the cannula component of an exemplary embodiment of the surgical access assembly according to aspects of the present invention in a misaligned configuration;

FIG. 2B is a cross section of the top portion of the pin component an exemplary embodiment of the surgical access assembly according to aspects of the present invention in an aligned configuration;

FIG. 2C is a top view of the pin component an exemplary embodiment of the surgical access assembly according to aspects of the present invention in a misaligned configuration;

FIG. 2D is a top view of the pin component of FIG. 2C in an aligned configuration;

FIG. 3 is a cross section of another exemplary embodiment of the pin component according to aspects of the present invention;

FIG. 4 is a perspective view of an exemplary control element for the pin component of FIG. 3;

FIG. 5 is a top view of the pin component of FIG. 2A;

FIG. 6 is a perspective view of the connector portion of the pin component of an exemplary embodiment according to aspects of the present invention;

FIG. 7 is a cross section view showing the torsional engagement between the pin component and cannula component of an exemplary embodiment according to aspects of the present invention;

FIG. 8 is a perspective view of an exemplary cannula component holder fastened to an exemplary cannula component according to aspects of the present invention;

FIG. 9 is a side view of the exemplary cannula component holder and cannula component of FIG. 8; and

FIG. 10 is a perspective view of the exemplary cannula component holder of FIG. 8.

It should be understood that the drawings are not necessarily to scale and that the disclosed embodiments are sometimes illustrated diagrammatically and in partial views. In certain instances, details which are not necessary for an understanding of the disclosed methods and apparatuses or which render other details difficult to perceive may have been omitted. It should be understood, of course, that this disclosure is not limited to the particular embodiments illustrated herein.

### DETAILED DESCRIPTION

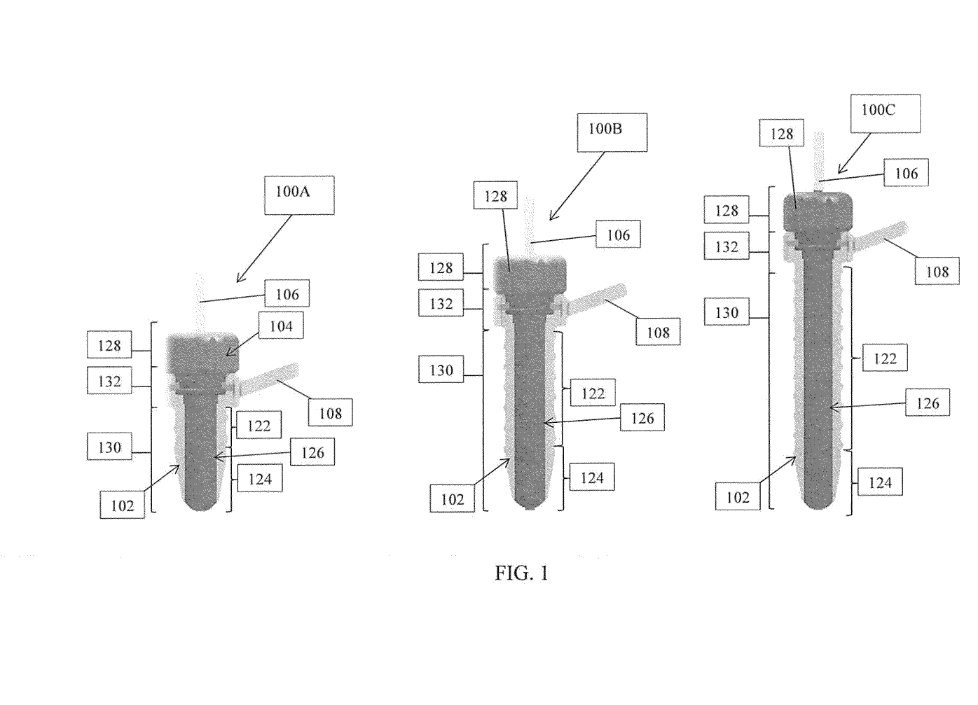

FIG. 1 illustrates a plurality of embodiments of the surgical access assembly of the present invention: surgical access assemblies 100A, 100B, and 100C. Surgical access assemblies 100A, 100B, and 100C (collectively, “surgical access assemblies 100” or “surgical access assembly 100”) preferably comprise cannula component 102, pin component 104, and handle component 108. In one embodiment, surgical access assemblies 100 are configured to receive and retain guide-pin component 106, which can be attached to standard navigation computer guidance systems (not shown), for example Cranial Navigation Probe, model number 9730269, sold under the brand name Microscope Probe by Medtronic Navigation of Louisville, Colo. In one embodiment, the navigation computer guidance system provides location information of the surgical area to guide the user during initial placement of a particular surgical access assembly. Use of a navigation system helps to ensure that the surgical access assembly is appropriately inserted to reveal the desired surgical area. A secured retention of guide-pin component 106 provides an accurate reading of the position of surgical access assemblies 100A, 100B, and 100C when a navigation system is used.

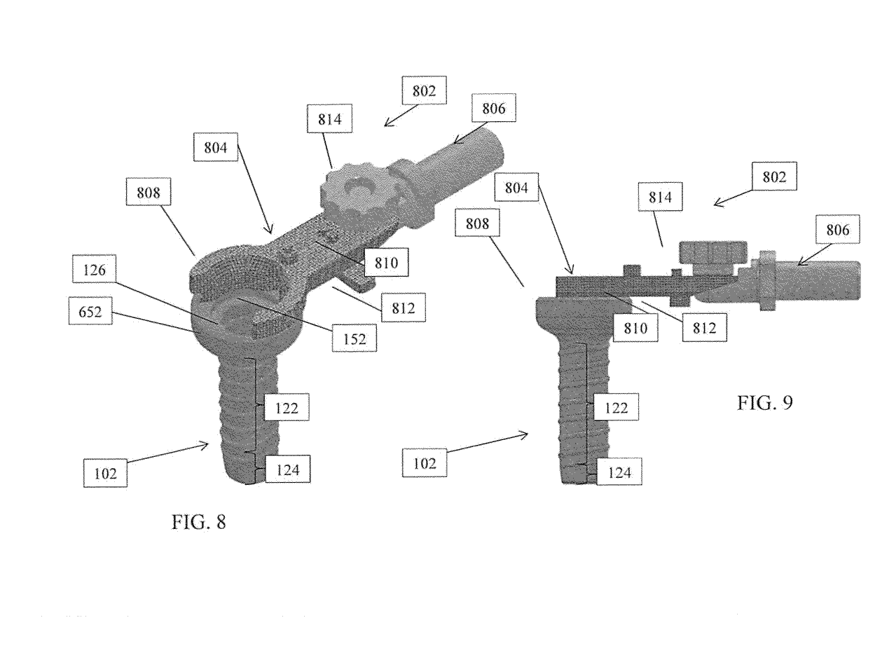

Referring to FIG. 1, pin component 104 is disposed in cannula component 102. As shown, cannula component preferably comprises inner cavity 126 that has a shape that engages and complements the shape of pin component 104. FIG. 8 also shows a perspective view of inner cavity 126. Referring to FIG. 1, inner cavity 126 preferably extends through cannula component 102 to provide an opening at the bottom of cannula component 102. After at least one surgical access assembly 100 is placed at the desired location in the target tissue, pin component 104 can be removed from cannula component 102 to expose inner cavity 126, thereby providing access to the target tissue at the desired location to a user for surgery or introduction of desired materials to that particular location. FIG. 8 shows cannula component 102 without pin component 104 and inner cavity 126 exposed. Inner cavity 126 preferably has a diameter configured to provide a sufficient work area at the desired location of the target tissue for the user. The diameter of inner cavity 126 can vary depending on the conditions of the particular surgical procedure. In one embodiment, the diameter of inner cavity 126 is sufficiently large to accommodate standard surgical instruments. As shown, surgical access assemblies 100 can be of various lengths to allow a user to reach the desired depth within the target tissue. The configuration of surgical access assembly 100 eliminates the need to “pull” a retractor against tissue portion to visualize a surgical area by initially providing a sufficient work area via inner cavity 126 of cannula component 102. Thus, the use of surgical access assemblies 100 eliminates or greatly lowers the possibility of accidental over-retraction with the use of conventional retractor systems because inner cavity 126 has pre-established dimensions. By avoiding excess retraction, damage to the surrounding tissues is also avoided, including possible brain damage.

Referring still to FIGS. 1 and 8, in the preferred embodiment, cannula component 102 preferably includes a plurality of surface features 122, preferably arranged like the threads on a screw, to allow surgical access assemblies 100 to be inserted into the target tissue by rotating surgical access assemblies 100. Cannula component 102 further includes tip portion 124, which is preferably tapered to facilitate the insertion of surgical access assemblies 100. Once placed in the targeted tissue, surface features 122 also help to anchor cannula component 102 to the target tissue, thereby assisting cannula component 102 to maintain the appropriate trajectory and/or access to the desired location within the target tissue.

Referring to FIG. 1, pin component 104 comprises head portion 128, body portion 130, and connector portion 132 disposed between head portion 128 and body portion 130. Head portion 128 preferably extends above cannula component 102. Body portion 130 is preferably generally cylindrical and has a length that is substantially the length of inner cavity 126 of cannula component 102. Connector portion 132 allows pin component 104 to releasably fasten to cannula component 102 and is described further in detail below. In the assembled configuration, body portion 130 and connector portion 132 are preferably disposed in cannula component 102.

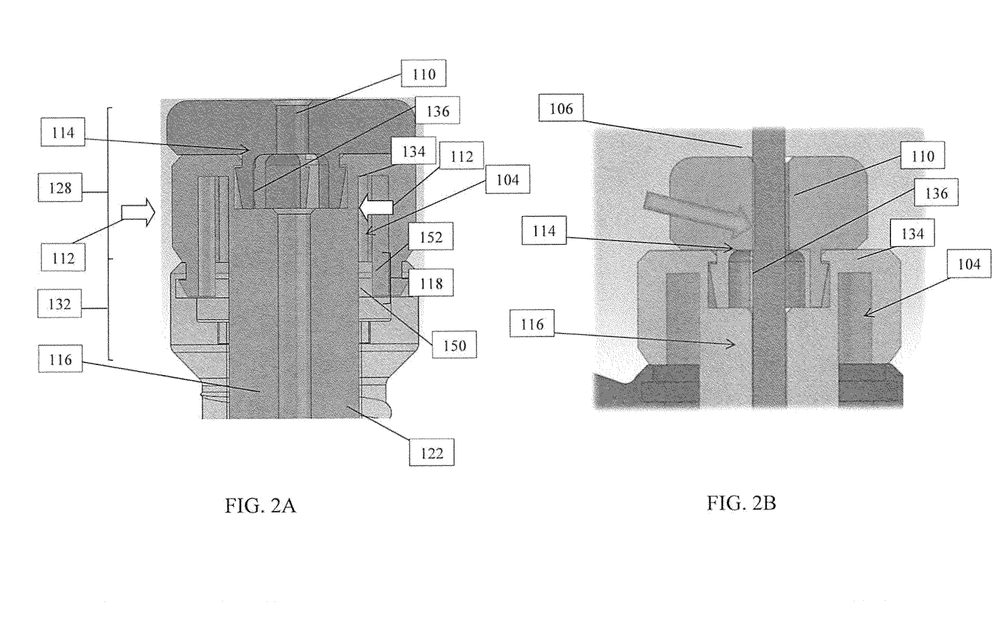

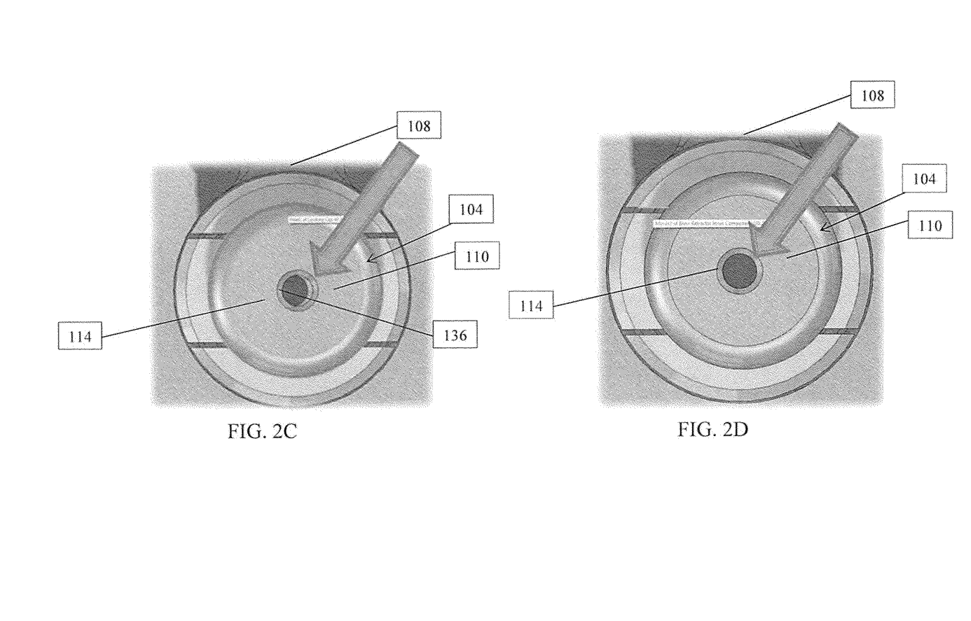

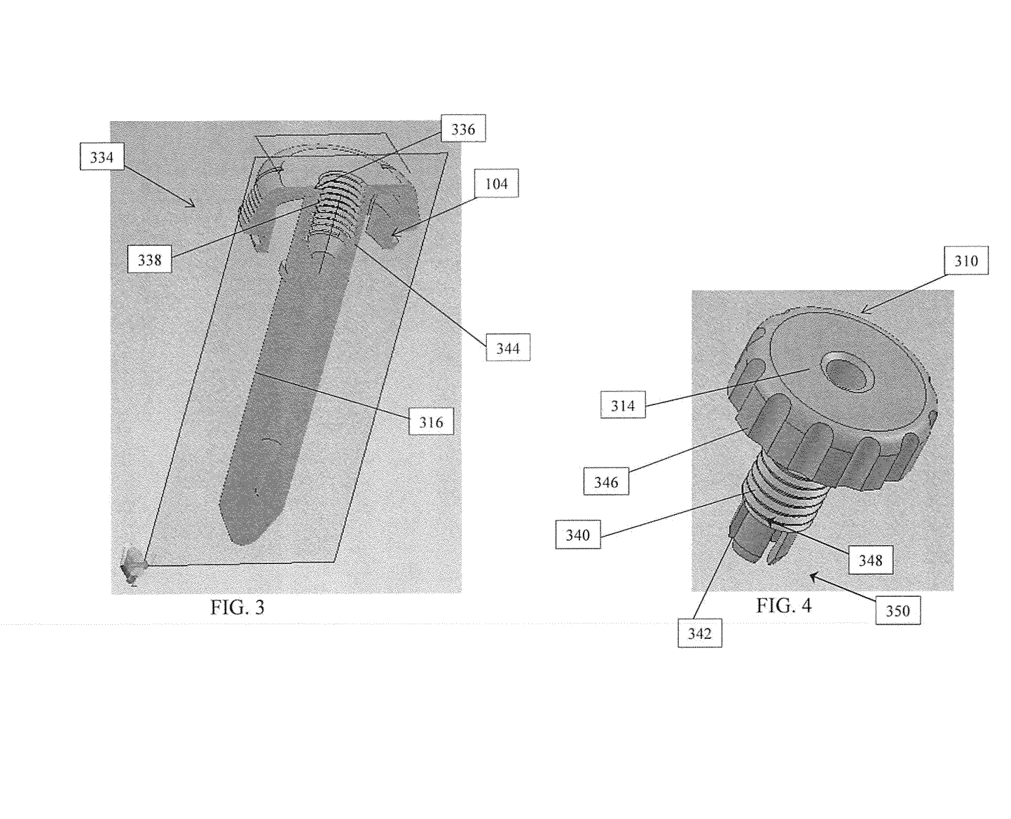

Referring to FIGS. 2A-2B, head portion 128 of pin component 104 comprises control element 110 preferably rotatably attached to tab element 134. Control element 110 further comprises channel 114 extending through control element 110. Channel 114 comprises adjustable section 136 that allows a user to control and adjust the effective or overall diameter of channel 114 by rotating control element 110. FIG. 2A shows adjustable section 136 misaligned, effectively narrowing the diameter of channel 114. Referring to FIG. 2B, when adjustable section 136 is in the aligned position, the effective diameter of channel 114 is at its maximum because adjustable section 136 aligns channel 114 with channel 116 of tab element 134. FIG. 2C is a top view of adjustable section 136 in the misaligned position, and FIG. 2D is a top view of adjustable section 136 in the aligned position. Referring to FIGS. 2A-2D, adjustments away from the aligned position narrows the effective diameter of channel 114. In one embodiment, guide-pin component 106 is inserted when adjustable section 136 is aligned with channel 116 and then secured by rotating control element 110 to the locked position where the misalignment of adjustable section 136 applies a gripping force against guide-pin component 106. This adjustable diameter arrangement also allows surgical access assemblies 100 to be used with guide-pin component 106 of different sizes. Channel 116 preferably extends from tab element 134 through connector portion 132 and at least a portion of body portion 130, similar to channel 316 as shown in FIG. 3. It is understood that channel 116 or 316 can be of any desired length.

FIGS. 3-4 show pin component 104 with an alternative embodiment of control element 310 and tab element 334 to securely retain a guide-pin component inserted into channel 314 and channel 316. In this embodiment, head portion 128 of pin component 104 comprises control element 310 rotatably attach to tab element 334. FIG. 3 shows a longitudinal cross section of pin component 102 with tab element 334 and without control element 310 rotatably attached. FIG. 4 shows a perspective view of control element 310. Referring to FIGS. 3-4, tab element 334 comprises generally cylindrical cavity 336 disposed preferably in the center of tab element 334. The dimension and shape of cavity 336 are configured to receive body 348 of control element 310. In a preferred embodiment, cavity 336 further includes a plurality of surface features 338 that are complementary to surface features 340 on body 348 of control element 310, where complementary surface features 338 and 340 are configured to allow control element 310 to rotatably engage with cavity 336 of tab element 334. Instead of having an adjustable section in the channel to apply a force to securely grip a guide-pin component, control element 310 comprises jaws 342 forming center channel 350. The distance between jaws 342, and thus the diameter of center channel 350, is adjustable through rotation of control element 310. When jaws 342 are tightened, the diameter of center channel 350 becomes more narrow and vice versa. The adjustability of jaws 342 allows for insertion of a guide-pin component and subsequent tightening of jaws 342 around the guide-pin component, providing a snug grip on the guide-pin component through rotation of control element 310. Referring to FIG. 4, in certain embodiments, control element 310 can further include indentations 346 to allow the user to have a better trip to rotate control element 310 in loosening or tightening jaws 342. The adjustability of jaws 342 through rotation of control element 310 can be achieved by mechanisms known to those of ordinary skill in the art. This adjustable diameter arrangement also allows surgical access assemblies 100 to be used with guide-pin component 106 of different sizes.

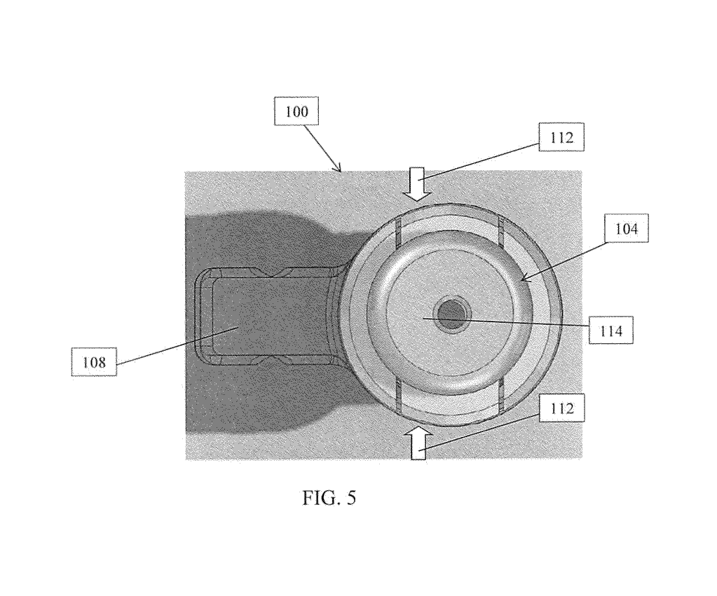

As mentioned above, pin component 104 is releasably fastened to cannula component 102. Referring to FIG. 2A, in one embodiment, the releasable fastening is achieved with latch engagement 118 between tab element 134 and cannula component 102. Latch engagement 118 includes latch portion 150 of pin component 104 and complementary latch portion 152 of cannula component 102. FIG. 3 provides a cross-sectioned perspective view of latch portion 344 of tab element 334, which is equally applicable to latch portion 150 of tab element 134. FIG. 8 shows a perspective view of latch portion 152 of cannula component 102. Referring to FIGS. 2A and 5, pin component 104 can be removed from cannula component 102 by applying an inward force to tab element 134 or 334, as indicated by arrows 112. Referring to FIG. 5, handle component 108 facilitates removal of pin component 104 by allowing the user to grip cannula component 102 to retain it in position while applying the inward and upward forces.

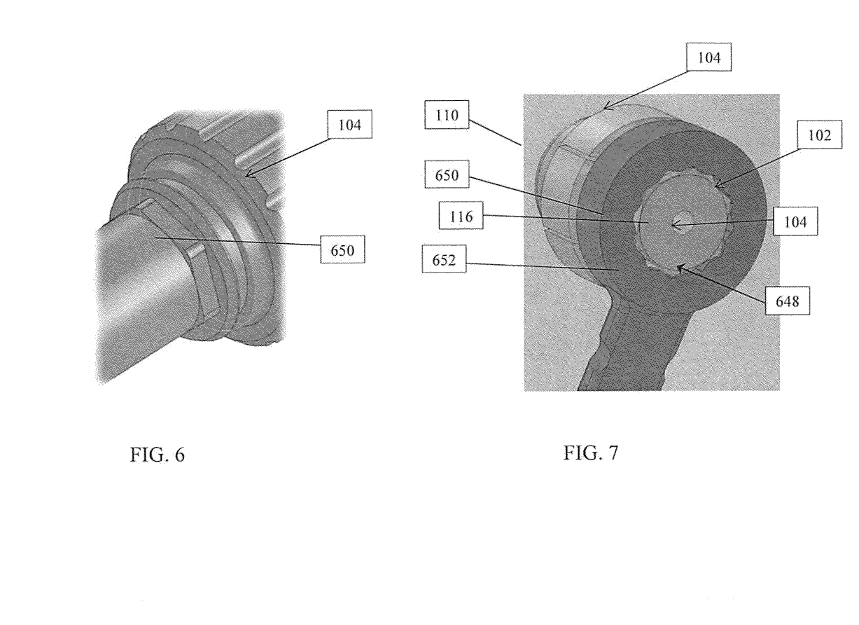

Connector portion 132 of pin component 104 further preferably comprises a torsional engagement that enables the translation of torsional forces between pin component 104 and cannula component 102. The preferred torsional engagement allows a user to rotate cannula component 102 into the target tissue by applying rotational force to head portion 128 of pin component 104. FIGS. 6-7 show one exemplary embodiment of the torsional engagement, torsional engagement 648, according to aspects of the present invention, which comprises a keyed hexagon drive.

Referring to FIG. 6, pin component 104 comprises hexagon shaped portion 650. FIG. 7 is a cross section view of the top of cannula component 102 coupled to hexagon shaped portion 650 of pin component 104. As shown, at least the portion of the wall of cannula component 102 surrounding hexagon shaped portion 650 has undulating surface 652 configured to engage and grasp hexagon shaped portion 650 such that any torsional force applied to pin component 104 is translated to also rotate cannula component 102 and vice versa. FIG. 8 provides a perspective view of undulating surface 652. Torsional engagement 648 allows a user to insert cannula component 102 into the target delicate tissue by rotating head portion 328 of pin component 104. FIGS. 6-7 merely show an exemplary embodiment of torsional engagement 648, which is not intended to be limiting. It is understood that torsional engagement 648 can include any mechanism that can translate torsional forces between pin component 104 and cannula component 102 known to those of ordinary skills in the art.

The dimensions of surgical access assembly 100 can vary and be modified according to an intended use. Generally, the surgical work space provided by cavity 126 of cannula component 102 preferably has a diameter in the range of approximately 8 millimeters (“mm”) to approximately 22 mm. The outer diameter of cannula component 102 preferably has a diameter in the range of approximately 12 mm. Particular dimensions of surgical access assembly 100 can be determined at least by the overall desired circumference and diameter for a particular use. Surgical access assembly 100 can be manufactured in a variety of useful sizes to be available as is practical.

Example dimensions of a surgical access assembly 100 in accordance with some embodiments, is provided in the table below:

| Width | Length |
|---|---|
| (Inner Diameter) | (Threaded Portion Only) |
| 8 mm | 3 cm |
| 8 mm | 5 cm |
| 8 mm | 7 cm |
| 8 mm | 9 cm |
| 12 mm | 3 cm |
| 12 mm | 5 cm |
| 12 mm | 7 cm |
| 12 mm | 9 cm |
| 16 mm | 3 cm |
| 16 mm | 5 cm |
| 16 mm | 7 cm |
| 16 mm | 9 cm |
| 22 mm | 3 cm |
| 22 mm | 5 cm |
| 22 mm | 7 cm |
| 22 mm | 9 cm |

In one embodiment, handle 108 is attached to cannula component 102 by compressing the tabs on arm element 804 whereupon locking arms move out and lock into the inner portion of undulating surface 652. Additionally, surgical bed frame attachment screw 814 may attach to the distal end of handle 108.

Surgical access assembly 100 can be formed of any biocompatible material which will provide sufficient stability and strength necessary to provide a surgical work area. The biocompatible material may be disposable or sterilize-able for repeated use. In one embodiment, surgical access assembly may be formed of a lightweight plastic material for ease of manipulation and/or the material may be transparent to allow direct visualization of underlying brain tissue thorough the instrument assembly portions. In one embodiment, cannula component 102 is preferably formed with clear polycarbonate. In another embodiment, surgical access assembly 100 is preferably sterile packed, either individually or in sets of specific quantities, for single-use purposes.

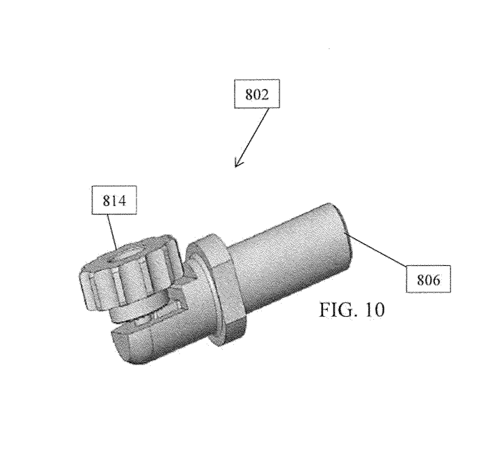

According to another aspect of the present invention, surgical access assembly 100 further includes a cannula holder component configured to connect to a frame or support system to maintain cannula component 102 in the desired position in the delicate tissue during and/or subsequent to the particular surgical procedure. FIGS. 8-10 illustrate a preferred embodiment of the cannula holder component of the present invention, indicated as cannula holder component 802. As shown, cannula holder component 802 comprises arm element 804 coupled to clip element 806. Arm element 804 preferably comprises circular portion 808 and body portion 810. Circular portion 808 is configured to engage and grasp cannula component 102. The tension of the grasp of circular portion 808 can be adjusted with trigger element 812 coupled to body portion 810. In some embodiments, trigger element 812 may include a ratcheting feature on the bottom that locks arm element 804 and body portion 810 into place.

Clip element 806 has one end that fastens to arm element 804 and another end that is adapted to receive or couple to any support rods that are part of a larger support or frame system configured to keep cannula component 102 in the desired position. In a preferred embodiment, clip element 806 is compatible with standard support or frame system. One exemplary manner that clip element 806 couples to body portion 810 via screw 814 extending through an opening (not shown) of body portion 810. Other known manners to couple fastener element 806 to arm element 804 can also be used. In one embodiment, clip element 806 is formed with stainless steel, allowing it to be reused with sterilization after each use, such as autoclave. Clip element 806 can be formed with any other suitable material that provides the desired strength and reusability. In one embodiment, the clip element 806 is made from 1385 stainless steel.

Certain embodiments of the surgical access assembly of the present invention can be used as follows. The following descriptions are merely exemplary and not intended to limit the present invention. An appropriately sized surgical access assembly is selected based on the surgical procedure and surgical site. It is then removed from the sterile packaging. A guide-pin component from a navigation system is inserted and secured to pin component 104. For pin component 104 with control element 110 as shown in FIGS. 2A-2D, surgical access assembly 100 is preferably packaged with adjustable portion 136 of control element 110 already aligned so guide-pin component 106 can be readily inserted into channel 114. Once guide-pin component 106 is appropriately inserted, the user can rotate control element 110 until sufficient force is applied to guide-pin component 106 to secure it to surgical access assembly 100. For pin component 104 with control element 310 as shown in FIGS. 3-4, surgical access assembly 100 is preferably packaged with jaws 342 of control element 310 at their maximum distance away from each other so guide-pin component 106 can be readily inserted into channel 314. Once guide-pin component 106 is appropriately inserted, the user can rotate control element 310 until jaws 342 sufficiently grasp guide-pin component 106 to secure it in place. Surgical access assembly 100, while coupled to a navigation system (not shown) via guide-pin component 106, is inserted into the target delicate tissue beginning with tip portion 124, where rotational downward force is applied to pin component 104 to screw surgical access assembly 100 in place. Once surgical access assembly 100 is appropriately positioned, the user can apply inward force to tab element 134 to disengage and remove it from cannula component 102 while holding to handle component 108 to retain cannula component 102 during removal of pin component 104. Cannula component 102 remains in place to provide a working channel for the procedure to be performed, where the channel is sufficiently large to accommodate standard surgical instruments. In some embodiments the cannula component 102 may be seen as optional and it is attached after the retractor has been inserted into the brain tissue in case additional stabilization of the retractor is desired.

Although the embodiments of the present disclosure and their advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A device for retracting tissue, said device comprising:
a cannula component having a longitudinal length, a tapered tip portion, an inner surface forming an inner cavity and an outer surface, said outer surface including at least one surface feature adapted to provide retention force on a target location and retain the cannula component within tissue when inserted; and
a pin component having a head portion, a body portion and a connector portion, said pin component configured to be attached within the cavity of the cannula component using the connector portion during insertion of the device, said pin component further adapted to be detachably removed from the inner cavity of the cannula component to provide access to a target tissue area through the inner cavity.

2. The device of claim 1 wherein the at least one surface feature includes one or more threaded edges configured to retain the cannula component within the target location.

3. The device of claim 2 wherein the threads are configured to assist in rotatably driving the cannula component into the target location.

4. The device of claim 1 wherein the longitudinal length of the cannula component is selected to correspond to a desired depth of insertion into a target location.

5. The device of claim 1 wherein the head portion of the pin component extends beyond the longitudinal length of the cannula component when inserted.

6. The device of claim 5 wherein the head portion is configured to receive force for insertion of the device and translate the insertion force onto the cannula portion.

7. The device of claim 6 wherein the head portion is configured to receive rotational force and translate the rotational force onto a cannula portion having a threaded outer surface feature.

8. The device of claim 1 further comprising a latch engagement portion adapted to latch the cannula component and pin component within the inner cavity of the cannula component.

9. The device of claim 1 further comprising a handle component configured to receive removal force and translate that force onto the cannula component for removal of the device.

10. The device of claim 1 wherein the pin component includes a cavity configured to receive a guide pin.

11. The device of claim 10 wherein the pin component is configured to securely retain the guide pin.

12. The device of claim 11 wherein the pin component includes an adjustable opening to retain different sizes of guide pins.

13. A method for making a retractor device, the method comprising:
forming a cannula component with a longitudinal length, a tapered tip portion, an inner surface forming an inner cavity and an outer surface, said outer surface including at least one surface feature adapted to provide retention force on a target location and retain the cannula component within tissue when inserted; and
forming a pin component with a head portion, a body portion and a connector portion, said pin component configured to be attached within the cavity of the cannula component using the connector portion during insertion of the device, said pin component further adapted to be detachably removed from the inner cavity of the cannula component to provide access to a target tissue area through the inner cavity.

14. The method of claim 13 wherein the at least one surface feature includes one or more threaded edges configured to retain the cannula component within the target location.

15. The method of claim 13 wherein the longitudinal length of the cannula component is selected to correspond to a desired depth of insertion into a target location.

16. The method of claim 13 wherein the head portion of the pin component extends beyond the longitudinal length of the cannula component when inserted.

17. The method of claim 16 wherein the head portion is configured to receive force for insertion of the device and translate the insertion force onto the cannula portion.

18. The method of claim 13 further comprising forming a latch engagement portion adapted to latch the cannula component and pin component within the inner cavity of the cannula component.

19. The method of claim 13 further comprising forming a handle component configured to receive removal force and translate that force onto the cannula component for removal of the device.

20. The method of claim 1 further comprising forming the pin component to include a cavity configured to receive a guide pin.

21. A method comprising:
providing a surgical access assembly having a cannula component and a pin component, the cannula component having a longitudinal length, a tapered tip portion, an inner surface forming an inner cavity and an outer surface, said outer surface including at least one surface feature adapted to provide retention force on a target location and retain the cannula component within tissue when inserted, the pin component having a head portion, a body portion and a connector portion, said pin component configured to be attached within the cavity of the cannula component using the connector portion during insertion of the device;
inserting the surgical access assembly at a target location;
removing the pin component from the cannula component thereby providing access to a target tissue area through the inner cavity of the cannula component.

22. The method of claim 21 further comprising attaching the surgical access assembly to a guide pin for insertion.
